(19) United States
(12) Patent Application Publication
(10) Pub. No.: US 2010/0304398 A1
(43) Pub. Date: Dec. 2, 2010

(54) NOVEL DIAGNOSTIC SENSOR FOR RAPID AND REPRODUCIBLE RO52 PROTEIN DOMAIN DETECTION

(75) Inventors: Ola Winqvist, Uppsala (SE); Marie Wahren-Herlenius, Djursholm (SE); Lars Baltzer, Uppsala (SE)

Correspondence Address: DARBY & DARBY P.C., P.O. BOX 770, Church Street Station, New York, NY, 10008-0770, US

(73) Assignee: MEADOWLAND BUSINESS PARTNERS AB, Mora (SE)

(21) Appl. No.: 12/532,195

(22) PCT Filed: Mar. 21, 2008
(86) PCT No.: PCT/IB2008/001470

§ 371 Date: Aug. 20, 2010

Related U.S. Application Data
(60) Provisional application No. 60/896,260, filed on Mar. 21, 2007.

(51) Int. Cl. G01N 33/53; G01N 33/566

(52) U.S. Cl. 435/7.1; 436/501

(57) ABSTRACT

The present invention relates to the use of specific synthetic sensor molecules for the discrimination of proteins and protein domains involved in autoimmunity. More specifically, in one embodiment, the invention relates to the detection of antibodies which bind to specific domains of the Ro52 protein. In another embodiment, the invention relates to the use of specific synthetic sensor molecules to identify domains of the Ro52 protein with different antibody specificities. The invention also includes a method for assessing the risk that a fetus will develop congenital heart block. The invention enables the evaluation and differential diagnosis of a range of autoimmune disorders, allowing appropriate treatment or more generally medical intervention decisions to be made.

This application claims priority to U.S. Provisional application 60/896,260, filed Mar. 21, 2007, which is incorporated by reference in its entirety.

### FIELD OF THE INVENTION

The present invention relates to the use of specific synthetic sensor molecules for the discrimination of proteins and protein domains involved in autoimmunity. More specifically, in one embodiment, the invention relates to the detection of antibodies which bind to specific domains of the Ro52 protein. In another embodiment, the invention relates to the use of specific synthetic sensor molecules to identify domains of the Ro52 protein with different antibody specificities. The invention also includes a method for assessing the risk that a fetus will develop congenital heart block. The invention enables the evaluation and differential diagnosis of a range of autoimmune disorders, allowing appropriate treatment or more generally medical intervention decisions to be made.

### BACKGROUND OF THE INVENTION

Predictive diagnostics is founded on the observation that variable clinical responses of patients to a drug are caused not only by physiological regulatory mechanisms and environmental factors, but also by their genetic constitution. The genetic profile of an individual determines the characteristics of the disease state as well as drug interaction with drug targets and other molecules in related biochemical pathways (the systems that regulate the body's functions). These characteristics in turn affect the individual's symptoms and responses to medical treatment(s). Therefore, through a better understanding of the correlation between complex biomarkers, effects on early diagnosis, drug targets and a patient's clinical response to drug treatment, predictive diagnostics has the potential to predict and define a patient's likely response to treatment with different classes of drugs, consequently improving treatment.

Whereas predisposition testing is restricted to identifying the risk of developing a particular disease, predictive diagnostics has a direct application in improving therapy choices for individuals who have already been diagnosed with a disease or disorder. Predictive diagnostics can be applied in therapies based on existing drugs as well as in support of the development of new drug molecules and treatment regimens.

Rather than solely predicting a disease or disorder, predictive diagnostics also facilitates the best choice of treatment or solution to a known disease or disorder. Predictive diagnostics therefore has a direct clinical utility.

### The Multivalent Ro52 Target

Autoantibodies directed toward both of the intracellular Ro/SSA antigens, comprising both the Ro52 and Ro60 protein components, and the La/SSB antigens are known to be associated with the adult rheumatic diseases Sjögren's syndrome and systemic lupus erythematosus (SLE). However, these autoantibodies may also be found in asymptomatic individuals. During pregnancy these autoantibodies are transferred to the fetus where they are implicated in the development of congenital atrio-ventricular (AV) heart block, and other disorders such as neonatal lupus erythematosus leading to dermatological, liver and hematopoeitic manifestations (See, for example, Wahren-Herlenius, M. and Sonesson, S. E., Specificity and effector mechanisms of autoantibodies in congenital heart block, Curr. Opin. Immunol. 2006, 18(6):690-6).

Congenital heart block is a transferred autoimmune disease that affects children whose mothers have autoantibodies reactive to the Ro/SSA and La/SSB antigens. Congenital heart block occurs in 2-5% of Ro/La positive pregnancies, and usually develops within the 18th-24th week of gestation. The mortality of fetuses diagnosed with complete congenital heart block is high, 10-30%, and around two thirds of children born with congenital heart block require the lifelong use of a pacemaker. The mechanism behind the development of congenital heart block is not fully understood, but it is known that isolated congenital heart block without associated structural malformations of the heart is almost always correlated to the presence of maternal Ro/SSA and La/SSB autoantibodies. In fetuses affected by the condition, there are signs of inflammation in the cardiac tissue with mononuclear cell infiltration, antibody and complement deposition. Autoantibodies to both Ro/SSA and La/SSB have been documented to associate with congenital heart block, but antibodies that recognize and are reactive toward the Ro52 protein component of the antigen show the strongest correlation with the development of congenital heart block. The strongest correlation is in those women in whom the antibody response is directed to amino acids 200-239 (p200) of the Ro52 kDa protein. In a prospective study in which Doppler echocardiography was performed weekly during susceptibility weeks 18-24 in Ro52-positive women, mothers with higher anti-p200 levels had fetuses with significantly longer atrioventricular time intervals (Salomonsson, S. et al., Ro/SSA autoantibodies directly bind cardiomyocytes, disturb calcium homeostasis, and mediate congenital heart block, J. Exp. Med., 2005, 201: 11-17).

The function of the Ro52 protein has not been fully established, although a role in ubiquitination and other regulatory processes has been proposed. Ro52 includes several predicted functional domains; two zinc-finger motifs are situated in the N-terminal region and a SPRY-region is near the C-terminus. The central part of Ro52 consists of a coiled-coil region, including a leucine zipper comprising amino acid (aa) residues 200-232. Leucine zippers, which contain periodic repeats of leucine amino acids every seventh residue, give rise to a helical structure, and are likely to be of importance for the correct folding of the protein, as well as its interaction with other molecules.

Based on an analysis of sequence similarity, the 475 amino acid (aa) protein Ro52 belongs to the tripartite motif (TRIM) family, also known as the Ring/B-box/coiled-coil family. The common motifs in this protein family comprise an N-terminal RING-type zinc finger, followed by a B-box-type zinc finger and a coiled-coil region spanning the central part of the protein. The coiled-coil region includes a putative leucine zipper contained within aa residues 211-232 of the coiled-coil region. Because each complete turn in an α-helix involves 3.6 amino acid residues, the leucine amino acids in the zipper α-helix appear near every second turn and face the same direction. This placement has been shown to optimize interactions with other zipper stretches. Both coiled-coils and leucine zippers are involved in homo- and hetero-multimerization and are crucial for protein-protein interactions in many biological systems. The C-terminal portion of the Ro52 protein has a high degree of similarity to the B30.2 or SPRY domain, which has not been clearly associated with a specific biological function.

It has been reported that the presence of maternal autoantibodies directed against the leucine zipper region of Ro52 is associated with the development of congenital heart block in children. Ro/SSA-positive mothers of children affected by fetal heart block showed a high level of antibodies directed against the 200-239 amino acid stretch of Ro52, including the leucine zipper motif, whereas Ro/SSA-positive mothers of healthy children had a predominant antibody reactivity against the 176-196 amino acid stretch of Ro52.

Recent studies indicate that antibodies recognizing the Ro52 protein of the Ro/SSA complex are pathogenic (Sonesson S. E., et al., Signs of first-degree heart block occur in one-third of fetuses of pregnant women with anti-SSA/Ro 52-kd antibodies, Arthritis Rheum. 2004, 50(4):1253-61), and more specifically, our studies have demonstrated that antibodies to amino acids 200-239 (p200) of the Ro52 protein were detected in the mothers of children with complete heart block (Salomonsson, S., et al., A serologic marker for fetal risk of congenital heart block, Arthritis Rheum. 2002, 46(5):1233-41). However, the fine specificity and mechanism by which p200-specific antibodies mediate congenital heart block have not been elucidated. Two human anti-Ro52 monoclonal antibodies have been cloned and characterized from SLE patients (Salomonsson, S., et al., Cloning and characterization of two human Ro52-specific monoclonal autoantibodies directed towards a domain associated with congenital heart block, J. Autoimmun. 2004 March; 22(2):167-77). These antibodies were shown to be directed against different epitopes within the 40-amino acid α-helical Ro52 p200 peptide (Ottosson L., et al., Structurally derived mutations define congenital heart block-related epitopes within the 200-239 amino acid stretch of the Ro52 protein, Scand. J. Immunol. 2005, 61(2):109-18). One of the human monoclonal antibodies, S3A8, was found to bind to the cell surface of cultured cardiomyocytes and induce the disregulation of intracellular calcium levels leading to cell death, while a second antibody, M4H1, did not. These findings demonstrate that only specific p200 reactive antibodies with a defined specificity bind to and functionally disable cardiomyocytes (Salomonsson, S. et al., Ro/SSA autoantibodies directly bind cardiomyocytes, disturb calcium homeostasis, and mediate congenital heart block, J. Exp. Med., 2005, 201: 11-17).

We and others have shown that early treatment of an incomplete heart block with high dose fluorinated steroids or corticosteroids prevents progression of, or even reverts, the block, decreasing fetal morbidity and mortality. However, a complete third-degree heart block is permanent, making it relevant also from a clinical point of view to define the specific antibody mediating congenital heart block. A marker with high predictability would identify high risk pregnancies and allow initiation of treatment at the critical stage to prevent irreversible heart block in the fetus.

Traditional assay detection systems, such as the enzyme-linked immunosorbent assay (ELISA), are inappropriate for modern screening needs due to their low sensitivity, requirement of multiple reagents and steps and variable reproducibility. The need for a faster, more sensitive and reproducible assay and detection system that is compatible with modern screening methods, such as micro-devices, is required. All publications, patents, and patent applications cited herein, whether supra or infra, are hereby incorporated by reference in their entirety.

### SUMMARY OF THE INVENTION

The present invention provides a method for assessing a risk that a fetus will develop congenital heart block comprising screening a bodily fluid of a woman in need of screening for the presence of an antibody specifically recognizing Ro52 p200 peptide. The method comprises contacting a bodily fluid of the woman, which may be serum, plasma, whole blood, amniotic fluid, cerebrospinal fluid, with an immobilized sensor molecule comprising a ModPro scaffold bearing a Ro52 p200 peptide, which has an amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, and SEQ ID NO: 18, and a fluorophore for a time period sufficient to allow any of the antibody that may be present in the fluid to bind to the molecule and thereby alter the fluorescence intensity of the fluorophore. The fluorescence intensity in the fluid, which may increase or decrease, is then compared to that of a control sample devoid of any antibody, and the difference in the fluorescence intensity correlates with the risk of heart blockage.

In another embodiment, the invention provides a method for detecting the presence of an antibody recognizing specifically Ro52 p200 peptide in a bodily fluid of a human. The method comprises contacting this fluid, which may be serum, plasma, whole blood, amniotic fluid, cerebrospinal fluid, with an immobilized sensor molecule comprising a ModPro scaffold bearing a Ro52 p200 peptide, which has an amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, and SEQ ID NO: 18, and a fluorophore for a time period sufficient to allow any of the antibody that may be present in said fluid to bind to the molecule and thereby alter the fluorescence intensity of said fluorophore, and comparing the fluorescence intensity in the fluid, which may increase or decrease, to that of a control sample devoid of the antibody.

In a further embodiment, the invention provides a method for assessing a risk that a fetus will develop congenital heart block comprising screening a bodily fluid of a woman in need of screening for the presence of an antibody specifically recognizing one or more Ro52 peptides. The method comprises contacting a bodily fluid of the woman, which may be serum, plasma, whole blood, amniotic fluid, cerebrospinal fluid, with a plurality of immobilized sensor molecules, each comprising a ModPro scaffold bearing an Ro52 peptide, which has an amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, and SEQ ID NO: 18, and a fluorophore for a time period sufficient to allow any of the antibody that may be present in the fluid to bind to the molecule and thereby alter the fluorescence intensity of said fluorophore; and comparing the fluorescence intensity of each sensor molecule, which may increase or decrease, to that of each of the other sensors.

### BRIEF DESCRIPTION OF THE FIGURES

FIG. 1 shows a schematic overview of a ModPro synthetic sensor molecule.

FIG. 2 shows a representation of the use of ModPro synthetic sensor molecules for the specific detection of the target under investigation.

FIG. 3 shows the entire amino acid sequence of human Ro52, also known as tripartite motif-containing 21, TRIM21 (Genbank NP_{—}003132.2; SEQ ID NO: 1).

FIG. 4 shows the result from the ModPro (top) and ELISA (bottom) 3 runs on the 36 clinical samples from Karolinska Institute, Solna, Sweden. Each point represents an average of the duplicates in each run.

### DETAILED DESCRIPTION OF THE INVENTION

### The ModPro Detection Principle

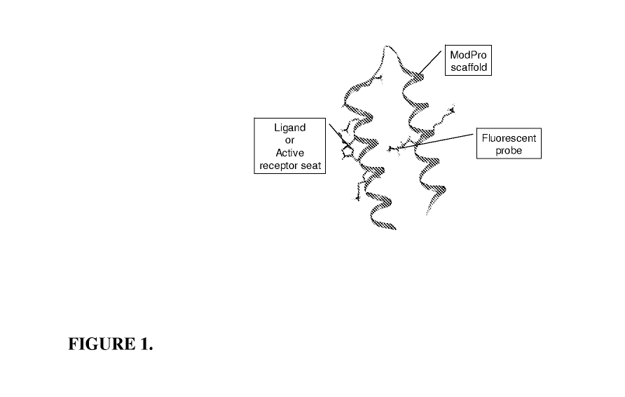

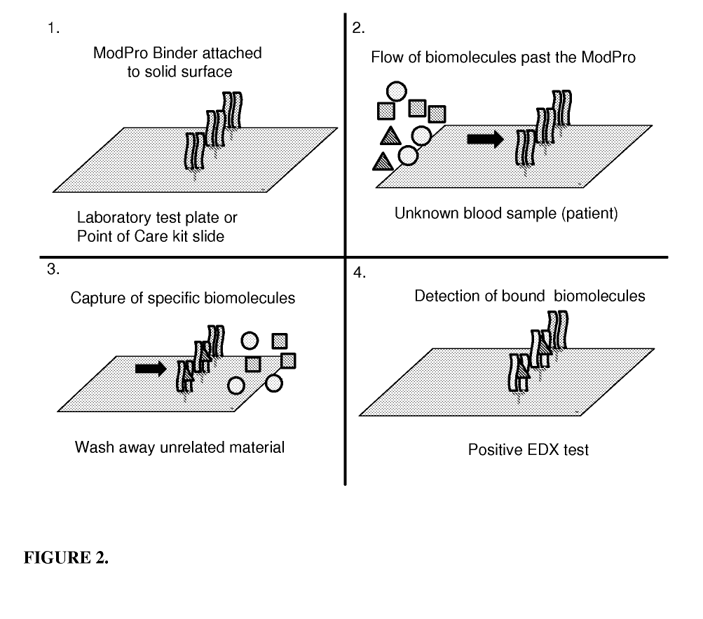

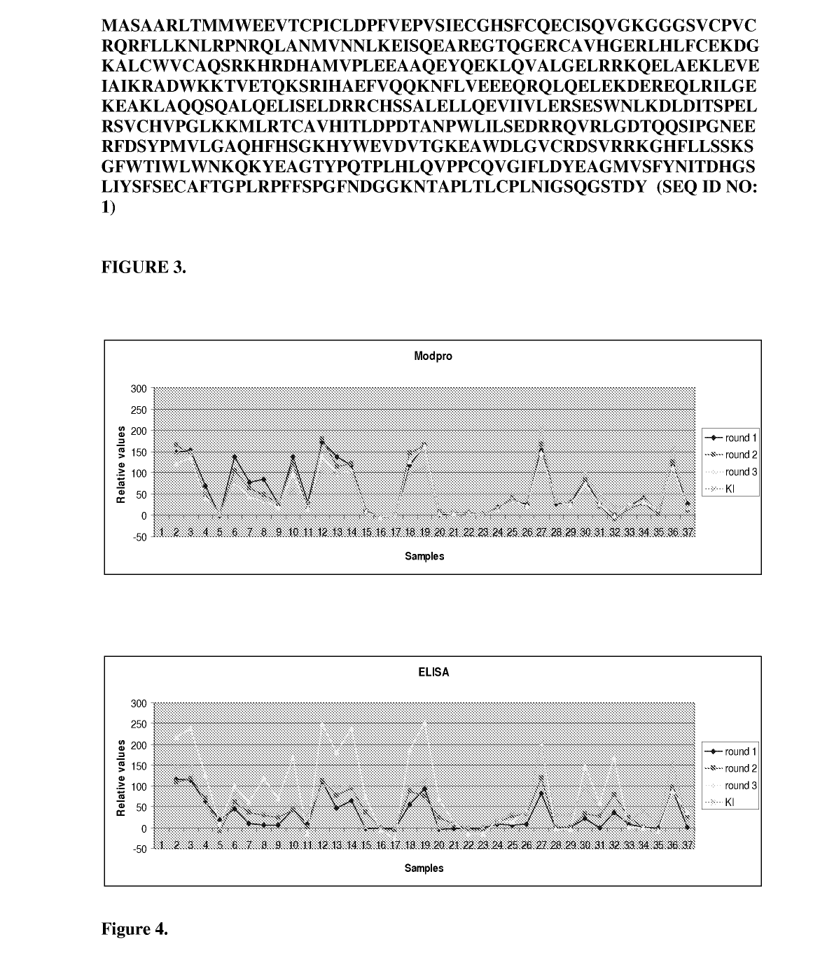

The ModPro scaffold and detection system, and related technologies, have been described in U.S. Pat. No. 6,171,810, U.S. Patent Publication Nos. 2005/0245727 and 2006/0234291, and PCT Publication WO03/044042, which are herein incorporated by reference in their entirety. The basic functionalized ModPro protein scaffold (see FIGS. 1 & 2) is formed by reaction of the protein with activated esters in aqueous solution, in order to form a stable amide bond site-selectively on the side chain of lysine residues. A molecule can be incorporated at one position while a fluorescence probe that can function as a detectable fluorophore is incorporated at another position. These reactions are typically carried out at room temperature and pH 6, are very robust and insensitive to variations in either or both pH and temperature. Active esters include any ester with a leaving group pKa in the range from 4-9. In addition, site selectivity is controlled through the manipulation of lysine pKa values, by incorporating them in positions on the ModPro protein scaffold that are hydrophobic. Since the four-helix bundle motif is based on the use of amphiphilic helices that fold due to hydrophobic interactions between the hydrophobic faces of the helices, the introduction of a lysine residue in the hydrophobic core provides a strategy to decrease the pKa of that lysine and to make it more reactive than any other lysine in the amino acid sequence.

The combination of these two chemical strategies makes it possible to construct proteins that carry several substituents in specific positions without intermediate purification that makes this protein scaffold unique, and extremely suitable for automation.

In one example of a protein target-ligand interaction, a ligand with known affinity is first attached onto a reactive amino acid side chain residue on the ModPro scaffold at a specific site (See, for example, Andersson, T., et al., Cooperative binding of human Carbonic Anhydrase II by functionalized folded polypeptide receptors, Chem. Biol., 2005, 12, 1245-1252). A fluorescent probe (fluorophore) is also attached, but on a different amino acid residue on the ModPro scaffold, thereby creating a functionalized biosensor that will both specifically recognize and become more (or less) fluorescent when bound to the target. In an alternative embodiment of the invention, the binding between the target and ligand of ModPro can be detected using a secondary antibody linked to an enzyme (such as alkaline phosphatase or horseradish peroxidase-coupled secondary antibodies; commercially available), such as is done in “sandwich” ELISAs.

The target can be a protein, antibody, antigen or any specific binding partner. The embodiment of linking the antigen to a ModPro and enhancing the antibody-antigen interaction by the general “stickiness” of the ModPro is envisioned to be beneficial with all applications when the target molecule is a specific antibody. It should be noted that it is generally possible to choose among a number of attachment sites. However, different attachment sites may confer different properties to the construct, as will be appreciated by those skilled in the art.

In the absence of the target protein the fluorescent probe (or “fluorophore”) will exhibit a low-level background intensity. When the target protein is added to the solution containing the modified MODPRO scaffold, such as by the addition of a biological sample containing the target, the fluorescence intensity changes relative to an untreated (or blank) solution, thereby signaling the binding and detection of the target protein. The alteration in fluorescence intensity is due to a change in the local environment of the fluorophore upon binding of the target molecule, and can lead to either an increase or decrease in fluorescence intensity. The addition of an inhibitor with sufficient affinity for the target protein would reverse the binding interaction, and this reversal in binding would be detected by an opposite change in fluorescence intensity relative to an untreated control.

The fluorescence detection capabilities imparted by the ModPro scaffold allow a number of unique advantages. First, the detection method does not require the addition of external labels, either primary or secondary, or substrate that may alter the intensity of the detection signal from experiment to experiment, improving the speed, sensitivity and reproducibility of the assay. However, it is noted that embodiments involving secondary antibodies are also encompassed by the instant invention. Secondly, the fluorescence detection is sensitive and applicable to miniaturization, such as incorporating the assay into micro-devices, and the like. Such miniaturization is an advantage for example in bringing the instant invention to the clinic or doctor's office.

Many fluorescent probes are commercially available from the SIGMA chemical company (Saint Louis, Mo.), Molecular Probes (Eugene, Oreg.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie A G, Buchs, Switzerland), and Applied Biosystems (Foster City, Calif.), as well as many other commercial sources known to one of skill. Furthermore, those of skill in the art will recognize how to select an appropriate fluorophore for a particular application and, if it not readily available commercially, will be able to synthesize the necessary fluorophore de novo or synthetically modify commercially available fluorescent compounds to arrive at the desired fluorescent label.

All ModPro screens are designed using similar principles, but involve the following varying parameters:
1. Variation of the bioactive molecule or molecules.
   One or several natural or synthetic ligands, or one or several peptide sequences corresponding to an active target receptor seat, can be incorporated onto the ModPro scaffold.
2. Variation of the position of the bioactive molecule or molecules.
   There are at least six amino acid residues that can act as “handles” from which a bioactive molecule can be attached.
3. The choice of detection probe.
   A number of fluorescent and non-fluorescent molecular probes may be attached onto the ModPro scaffold.

ModPro is particularly useful in that any number of ligands or proteins may be selectively attached to it, thus enabling a multitude of tests to be carried out per chemical compound in combinatorial libraries. For example, the platform lends itself to a multiplex assay involving many antigens being evaluated simultaneously.

Peptides (for example hormones, signal peptides, inhibitors, antigens, antigen fragments, antibodies and antibody fragments), peptide nucleic acids, carbohydrates and enzyme inhibitors are examples of biomolecules that can be combined and used in a rational or combinatorial manner to form an array of binding sites. Components from combinatorial libraries may also be incorporated, as long as a corresponding active esters are available. Active esters may be synthesized by activating a carboxylic acid residue, oxidizing an alcohol or aldehyde group to a carboxylic acid, followed by activation, or direct oxidation of an unsaturated bond, such as an alkene or alkyne. These techniques are well known to one of skill in the art.

An especially preferred embodiment of the present invention is a ModPro scaffold comprising both a fluorescent probe and a ligand for natural protein, thus constituting a biosensor. If the affinities of the ligands are varied, an array for the determination of protein concentration can be constructed. If the receptor site mimics that of the biologically relevant one, then a tool for screening in drug development has been obtained. In addition, the ligands can be attached in a combinatorial way by reacting a large number of different ligands with the scaffold simultaneously.

The ModPro protein scaffold is modified to have a basic, acidic or a hydrophobic surface. The distance between, and the number of, attachment points is also modified, depending on the particular target. In one embodiment, the ModPro protein scaffold is attached to a solid support. The scaffolds therefore provide a flexible tool for the construction of artificial receptors, where the nature of the binding site is designed by the user. The scaffold chemistry also provides for the incorporation of peptide loops. This is achieved by exploiting the chemistry “twice.” First, one end of the peptide to be looped is attached to the most reactive position of the scaffold, then the other end is attached to the second most reactive site, at which point the process proceeds as described herein. The incorporation of loops of peptides or other polymers, enhances affinities of peptide or polymer receptors tremendously, and expands the versatility of the ModPro concept. There are several different strategies for the incorporation of loops, by way of example, is the synthesis of a peptide where the C-terminal carboxyl group is prepared in the form of an ester and the N-terminal residue is aspartic or glutamic acid where the side chain is prepared in the form of an ester.

Key advantages of the ModPro approach are cost of production, flexibility and ease of use. The usefulness of the scaffold is due to the fact that the relative reactivity of each attachment site determines the order of incorporation and that it is controlled entirely by the protein. The multivalent capacity of the protein allows it to direct a first ligand to a first binding site, a second ligand to a second binding site, etc., and is to our knowledge unique and not described in the prior art. The reactivity of the substrates determines the rates of incorporation.

The benefits of the ModPro scaffold in biological assays, and ModPro fluorescence detection, over traditional assay systems include a substantial improvement in binding affinity (low nM on a regular basis) with good selectivity. The synthetic methodology for functionalizing the scaffold is well-defined, and since the peptide-functionalized Mod-Pro scaffold can be chromatographically purified and characterized, it is well-suited to commercialization. It can also be produced in abundance without batch to batch variation. The system provides a robust and reproducible signal with low or non-existent interference, which enables its use in micro-device and micro-assay systems. In fact, as shown below, the ModPro assay is at least six times more effective than a traditional ELISA. The ModPro scaffold also provides a small size, higher light intensities per unit area and standardizability of the assay. In addition, by modifying the position of the binding peptide and fluorophore on the ModPro scaffold, and in relation to each other, it is possible to obtain tunable affinities and selectivities.

### A MODPRO Scaffold for the Recognition of Ro52 Autoantibodies

In one embodiment, the present invention is a novel, quantitative diagnostic tool. Novel combinations of synthetic sensor molecules with unique protein-motif binding capabilities clearly categorize and differentiate the different diseases of Sjögren's syndrome, SLE and congenital heart block. This is accomplished by detecting, mapping and quantifying the specific biochemical interactions that are mediated by different portions (epitopes) of the Ro52 protein, and the antibodies that bind these protein epitopes, using the specificity and unique multivalent functionalization of the ModPro synthetic binding scaffold. The Ro52-specific structural domains specify a number of biochemical interactions related to disease development and/or pre-symptomatic detection of disease states, for example autoantibody generation.

Autoantibodies, for example, directed against specific motifs of the Ro52 protein, such as p200, and specific sequences within p200, have shown a strong correlation with fetal heart block and the specific detection of these antibodies. Using the novel ModPro synthetic sensors of the present invention will facilitate the prediction and early intervention of those women whose fetus is at risk of developing congenital heart block. Using this new technology we have developed a tool for the detection, with extremely high specificity and reproducibility, of women at the highest risk of bearing a child suffering congenital heart block, which allows for an appropriate, early monitoring and/or treatment intervention.

The P200 sensor comprises the ModPro scaffold peptide to which the Ro52 p200 peptide has been attached. The resulting chemical entity defines the synthetic sensor molecule specifying the ligand for detection of Ro52 autoantibodies and hence, detection of women at risk for fetal heart block.

In an alternate embodiment, interactions involving motifs within the Ro52 protein are correlated with SLE and/or Sjögren's syndrome. Based on this discovery, a second embodiment of the present invention defines a new tool for the categorization and differentiation of the different diseases of Sjögren's syndrome, SLE and other autoimmune disorders, for example rheumatoid arthritis, by detecting, mapping and quantifying the specific biochemical interactions that occur with different parts (epitopes) of the Ro52 protein. The distinct illnesses specified above have different pathogeneses and thus require different treatments or palliative interventions to achieve optimal treatment efficacy for the patient. The invention will markedly enhance current clinical practice by allowing the determination of appropriate treatment regimes for each individual.

The assay subjects include women in need of screening, such as pregnant women, especially those in the early stages of pregnancy (prior to week 16), as well as non-pregnant women of childbearing age, or women who may have autoimmune antibodies but present with no symptoms of autoimmune pathology.

In an embodiment of the invention, a solid support such as a dipstick is coated with the P200 sensor ModPro molecules, allowing for the Ro52 detection assay to be done in the clinic or in the doctor's office.

### EXAMPLES

### Example 1

### The P200ModPro Sensor Molecule

Amino acid sequence of the ModPro scaffold:

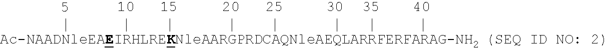

The ModPro scaffold peptide was synthesized using standard peptide synthesis techniques on solid phase resin. Before cleaving the peptide from the resin, Lysine 15 (L15) was deprotected over 3 h at room temperature with [Pd(PPh₃)₄] (3 equiv) in a mixture of trichloromethane, acetic acid and N-methylmorpholine (17:2:1 v/v; 12 mL per g of polymer). The resin was washed sequentially with 20 mM diethyldithiocarbamic acid in DMF, 30 mM diisopropylethylamine (DIPEA) in dimethylformamide (DMF), DMF and dichloromethane (DCM), and then desiccated. The fluorophore 7-methoxy-coumarin-3-carboxylic acid was then attached to the selectively deprotected lysine residue (L15) using a carbodiimide coupling reaction, which is well known to persons skilled in the art. The Ro52 peptide p200 is coupled through its amino terminus to the side chain of glutamate 8 (E8) using native chemical ligation (Dawson, P. E., et al., Synthesis of proteins by native chemical ligation, Science, 1994, 266(5186), 776-9). Both the E8 and L15 amino acids are marked boldfaced and underlined in the sequence above. Amino acid cysteine 24 (C24) is used to anchor the ModPro to a solid support and is protected by a acetamidomethyl group, Acm, until the coupling reaction is performed. Alternatively, the ModPro scaffold is bound to a solid support using other techniques known in the art, such as the biotin/streptavidin method. In this case, the peptide is modified conjugated to a biotin molecule, which has a high affinity the protein streptavidin (avidin). The biotin modified ModPro scaffold can then be adhered to any streptavidin-coated surface.

The Ro52 p200 peptide native structure:
(SEQ ID NO: 3)
H₂N-LEKDEREQLRILGEKEAKLAQQSQALQELISELDRRCHSS-CO₂H
The cysteine-modified Ro52 p200 peptide:
(SEQ ID NO: 4)
H₂N-CEKDEREQLRILGEKEAKLAQQSQALQELISELDRRAHSS-CO₂H

In the cysteine modified Ro52 p200 peptide, a leucine has been replaced with a cysteine and wild-type cysteine has been replaced with an alanine, and this is linked to the synthetic sensor scaffold molecule (ModPro) using native chemical ligation. (See, Nilsson B L, Soellner MB, Raines RT. 2005. Chemical Synthesis of Proteins. Annu. Rev. Biophys. Biomol. Struct. 34:91-118, for a review of native chemical ligation).

### The P200-Q8/E28 ModPro Sensor Molecule.

The Q8-E28 stretch of the Ro52 p200 peptide has been shown to be the target of the human monoclonal anti-Ro52 antibody S3A8 (Ottosson L., et al., Structurally derived mutations define congenital heart block-related epitopes within the 200-239 amino acid stretch of the Ro52 protein, Scand. J. Immunol. 2005, 61(2):109-18). This monoclonal antibody was found to bind to the cell surface of cultured cardiomyocytes and induce the disregulation of intracellular calcium levels leading to cell death (Salomonsson, S. et al., Ro/SSA autoantibodies directly bind cardiomyocytes, disturb calcium homeostasis, and mediate congenital heart block, J. Exp. Med., 2005, 201: 11-17). This finding demonstrates that the presence of antibodies with a defined specificity for the Q8-E28 segment of the Ro52 p200 peptide would provide evidence of a fetus at higher risk for developing congenital heart block. A P200-Q8/E28 ModPro sensor molecule is constructed as described above for the P200 ModPro sensor molecule, with the exception that the Ro52 p200 Q8/E28 peptide is coupled to the scaffold instead of the entire native structure. It is also envisioned that one or more modified Q8/E28 peptides will be synthesized and used to construct additional ModPro sensor molecules. Such modifications include, but are not limited, to cysteine modifications, such as the Ro52 p200C Q8/E28 peptide shown below, or alanine-scanning mutations where one or more amino acids of the native sequence are mutated to alanine. These modifications are expected, among other benefits, to allow alternate coupling pathways and peptides for both control assays and multiplex assays.

The Ro52 p200-Q8/E28 peptide native structure:
H₂N-QLRILGEKEAKLAQQSQALQE-CO₂H (SEQ ID NO: 5)
A modified Ro52 p200C-Q8/E28 peptide structure:
H₂N-CQLRILGEKEAKLAQQSQALQE-CO₂H (SEQ ID NO: 6)

### The P200-A25/S40 ModPro Sensor Molecule.

The human monoclonal anti-Ro52 antibody M4H1 has been shown to protect the A25-S40 stretch of the Ro52 p200 peptide from degradation (Ottosson L., et al., Structurally derived mutations define congenital heart block-related epitopes within the 200-239 amino acid stretch of the Ro52 protein, Scand. J. Immunol. 2005, 61(2):109-18). This monoclonal antibody was also found not to bind to the cell surface of cultured cardiomyocytes (Salomonsson, S. et al., Ro/SSA autoantibodies directly bind cardiomyocytes, disturb calcium homeostasis, and mediate congenital heart block, J. Exp. Med., 2005, 201: 11-17). A P200-A25/S40 ModPro sensor molecule is constructed as described above for the P200 ModPro sensor molecule, with the exception that the Ro52 p200 A25/S40 peptide is coupled to the scaffold instead of the entire native structure. It is also envisioned that one or more modified A25/S40 peptides will be synthesized and used to construct additional ModPro sensor molecules. Such modifications include, but are not limited, to cysteine modifications, such as the Ro52 p200C A25/S40 peptide shown below, or alanine-scanning mutations where one or more amino acids of the native sequence are mutated to alanine. These modifications are expected, among other benefits, to allow alternate coupling pathways and peptides for both control assays and multiplex assays.

The Ro52 p200-A25/S40 peptide native structure:
H₂N-ALQELISELDRRCHSS-CO₂H (SEQ ID NO: 7)
A modified Ro52 p200CA-A25/540 peptide
structure:
H₂N-CALQELISELDRRAHSS-CO₂ (SEQ ID NO: 8)

### Additional ModPro Sensor Molecules.

Sensor molecules comprising additional native and/or mutated peptide sequences of Ro52 are envisioned. The sensor molecules comprising these peptides are synthesized using the procedure described above for the P200 ModPro sensor molecule. A number of these peptides are exemplified below.

The Ro52 p197 peptide:
(SEQ ID NO: 9)
H₂N-LQELEKDEREQLRILGEKEAKLAQQSQALQELISEL-CO₂H
The Ro52 pZIP peptide:
(SEQ ID NO: 10)
H₂N-LVKDLREQLRILGEKVAKLAQQSQALQELISELDRRCHSS-CO₂H
The Ro52 pOUT peptide:
(SEQ ID NO: 11)
H₂N-LEKDERQQLRILGNKEAKLAQQSQALQKLISELDRRCHSS-CO₂H
The Ro52 pA233 peptide:
(SEQ ID NO: 12)
H₂N-LEKDEREQLRILGEKEAKLAQQSQALQELISELARRCHSS-CO₂H
The Ro52 pA234 peptide:
(SEQ ID NO: 13)
H₂N-LEKDEREQLRILGEKEAKLAQQSQALQELISELDARCHSS-CO₂H
The Ro52 pA235 peptide:
(SEQ ID NO: 14)
H₂N-LEKDEREQLRILGEKEAKLAQQSQALQELISELDRACHSS-CO₂H
The Ro52 pA236 peptide:
(SEQ ID NO: 15)
H₂N-LEKDEREQLRILGEKEAKLAQQSQALQELISELDRRAHSS-CO₂H
The Ro52 pA237 peptide:
(SEQ ID NO: 16)
H₂N-LEKDEREQLRILGEKEAKLAQQSQALQELISELDRRCASS-CO₂H
The Ro52 pA238 peptide:
(SEQ ID NO: 17)
H₂N-LEKDEREQLRILGEKEAKLAQQSQALQELISELDRRCHAS-CO₂H
The Ro52 pA239 peptide:
(SEQ ID NO: 18)
H₂N-LEKDEREQLRILGEKEAKLAQQSQALQELISELDRRCHSA-CO₂H

### Immobilization of the Functionalized ModPro Scaffold Molecules on a Solid Support.

The functionalized ModPro scaffold is then immobilized on a clean, optionally modified surface such as plastic, glass or metal. The surface can be a flat structure, such as a dipstick, or in the form of a particle or in the shape of a well.

The above principal description of the coupling of the synthetic sensor molecule to an analytical platform is included by way of example only and is not intended to limit the scope of the present invention.

The amino acid residue abbreviations used in the description of the present invention are as follows:

| | |
|---|---|
| L-Amino | |
| Acids | Abbreviations |

| | | |
|---|---|---|
| Alanine | Ala | A |
| Arginine | Arg | R |
| Asparagine | Asn | N |
| Aspartic Acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Nor-Leucine | N-leu | Nle |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

### P200ModPro Assay Protocols

### Indication for the Detection of Autoantibodies

Autoantibodies against Ro52 in women suffering from, for example, Sjögren's syndrome or SLE may cross the placenta during pregnancy and give rise to congenital heart block in the fetus. Detailed studies show that the Ro52 p200 peptide from the Ro/SSA autoantigen is the epitope which is recognized by the autoantibodies that may give rise to congenital heartblock (Ottosson, L. et al., Structurally derived mutations define congenital heart block-related epitopes within the 200-239 amino acid stretch of the Ro52 protein, Scand. J. Immunol. 2005, 61(2):109-18). These Ro52 p200 autoantibodies cross the placenta and react with an ion channel in the heart muscle of the fetus which may result in heart block. Congenital heart block occurs during week 18-24 of the development of the fetus. If discovered in time, it can be treated using steroids and prevent a lifelong need of a pacemaker or intrauterine death of the fetus.

Ro52 p200 autoantibodies are highly predictive for identifying women carrying a fetus (or who would carry a fetus) with a high risk of developing congenital heart block. The analysis is performed on sera, plasma, whole blood, amniotic fluid, or cerebrospinal fluid from fertile women with homogenous ANA-pattern, if autoantibodies against SS-A52 have been demonstrated or when clinically indicated.

### The P200 ModPro Autoantibody Assay

Antigen (P200 ModPro) is linked or coated to a pretreated, or chemically modified, microwell plate in order to form the ModPro assay plate. Pretreatment of the plate can be coating with streptavidin (avidin), such as in cases where the ModPro scaffold contains a biotin group. A number of commercially available pre-coated/pre-treated microtiter plates are also available (Pierce, Inc., Rockford, Ill., USA).

A blocking of the ModPro assay plate is used to reduce nonspecific binding to the microwell plate. Standard blocking agents such as non-fat milk or bovine serum albumin (BSA), or other agents known to those in the art, may be used. The patient sample is added to a microwell plate, and known controls of specific antibodies are added to separate microwell plates.

Antibodies that have reacted with the surface-bound antigen will remain attached to the plate and are detected with a rabbit anti-human IgG coupled to the enzyme alkaline phosphatase (AP). A color reaction occurs when the substrate is oxidized by the enzyme. The intensity of the color is measured using an spectrophotometer in which different filters are used to achieve a wave length suitable for the substrate.

### Reagents

| Coating Buffer | pH 9.6 | |
|---|---|---|
| Na₂CO₃ | 3.18 | g |
| NaHCO3 | 5.86 | g |
| NaN₃ | 1 | mL |
| Purified H₂O to 1000 mL | | |
| Substrate Buffer | pH 9.8 | |
| MgCl₂•6H₂O | 0.101 | g |
| C₄H₁₁NO₂ diethanolamine | 97 | mL |
| NaN₃ (20%) | 1.0 | mL |
| Purified H₂O to 1000 mL | | |
| Assay Buffer | pH 7.4 | |
| NaCl | 8.77 | g |
| NaH₂PO₄•H₂O | 0.90 | g |
| Na₂HPO₄•2H₂O | 7.74 | g |
| BSA (bovine serum albumin) | 2 | g |
| TWEEN 20 | 0.5 | mL |
| NaN₃ (20%) | 1 | mL |
| Purified H₂O to 1000 mL | | |

Microwell Plates
F96 CERT. MAXISORP
NUNC-Immuno plate

PBS
PHOSPHATE BUFFERED SALINE TABLETS (Sigma Cat No. P-4417, Milwaukee, Wis., USA)

Blocking, 4% BSA in PBS
Albumin, bovine serum (Sigma Cat No. A9647, Milwaukee, Wis., USA)

Coating Antigen
P200 ModPro 1 mg/mL (ModPro A B, Sweden)

Conjugate
Rabbit anti human IgG-AP
DAKO D0336 (1:500)

Substrate
Phosphatase tablets (Sigma Cat No. S16MA 50942-200TAB, Milwaukee, Wis., USA)

Washing Solution; Stock Solution

| | | |
|---|---|---|
| NaCl | 876.6 | g |
| NaH₂PO₄•H₂O | 4.48 | g |
| Na₂HPO₄•2H₂O | 38.71 | g |
| TWEEN 20 | 100 | mL |
| NaN₃ | 100 | mL |
| Purified H₂O | 5000 | mL |

Washing Solution; Working Solution
The stock solution is diluted 1:20 using purified H₂O in order to make up the working solution.

| | |
|---|---|
| REMITTANCE | Immunology |
| SAMPLE | Vacutainer tubes without any addition or Vacutainer |
| COLLECTION | tubes with gel. |
| SAMPLE | |
| MATERIAL | Serum. Analysis volume 1 mL. |
| | The blood is centrifuged and the serum separated from |
| | blood cells. |
| STORAGE | Refrigerator |
| CONTROLS | Low positive control |
| | High positive control |
| | Negative control |

### Experimental Protocol

(1) Blocking
The plate is washed with PBS.
The plate is blocked by adding 200 p14% BSA/PBS and incubated at room temperature for 1 hour.
The plate is then washed once with PBS.

(2) Coating of the Plate with P200ModPro or Control Antigen
A dilution of the P200ModPro (3 μg/ml) is made in coating buffer. 100 μl is added to each well and allowed to incubate for 1 hour at room temperature.
The plate is washed 4 times using the washing solution.

(3) Preparation of Control and Patient Samples
The control and patient samples are diluted 1:300 in Assay Buffer.

(4) Addition to the Plate
100 μl of the blank, diluted control (High positive control is added twice) and patient samples are added to the plate and allowed incubate on a shaker for 1 hour at room temperature.

(5) Addition of Conjugate
The anti-human IgG AP conjugate is diluted 1:500 in Assay Buffer
The plate is washed 4 times with washing solution.
100 μl of conjugate is added to each well.
The plate is incubated for 1 hour at room temperature on a shaker.

(6) Addition of Substrate
The substrate tablet is dissolved in 5 mL of substrate buffer (at least 10 min before use).
The plate is washed 4 times with washing solution.
100 μl of substrate is added to each well.
The plate is incubated at room temperature without shaking.

(7) Plate Reading
The plate is read using a spectrophotometer 405 nm, double wavelength (DW) when the absorbance for the high control is at least 1.5 nm.

| | |
|---|---|
| EVALUATION | Calculate Index according to the following: |
| | (ODₛₐₘₚₗₑ − OD_{negative control})/ |
| | (OD_{positive control (S61)} − OD_{negative control})) * 100 |
| REFERENCE | <25 not detected |
| VALUE | 25-40 slightly increased risk |
| | >40 highly increased risk |

Detection of Binding using Fluorescence

For embodiments of the present invention where binding of the ModPro scaffold to its target is measured using the fluorescence, the protocol is essentially the same as that described above, with the exception that conjugate/substrate are not required, and plate reading is performed using a spectrophotometer capable of reading fluorescence.

An observable difference in fluorescence (either an increase or decrease) indicates the presence of antibody and a positive risk for congenital heart block. A significant difference in fluorescence compared with a control sample known to be devoid of the antibody indicates the presence of a significant amount of antibody and a significantly elevated risk for congenital heart block.

### Example 2

### Optimization of the P200 ModPro Assay

The novel P200 ModPro sensor molecule examination method was developed and its robustness evaluated using a series of experiments that were planned according to predefined statistical experimental design schemes. Initially, a careful assessment was made concerning which experimental variables that could be considered to have an impact on the outcome of the test reaction. An experimental test plan with a series of tests was thereafter designed, in which all these parameters (variables) were systematically changed simultaneously in order to optimize the assay.

The overall objectives of this development project were to develop a novel method to identify P200-positive samples which is cheaper and faster but yet minimally as sensitive and reliable as the existing gold standard ELISA method.

The hypothesis was that the commercially available antibody binders of ELISA could be successfully substituted by the considerably smaller, more chemically stable and cheaper ModPro binder molecules. A secondary, but important, idea was also that incubation times potentially could be shortened and/or that one or more steps in the original ELISA could be merged, or even excluded. The basis for this assumption was essentially that the size of the ModPro allows for faster diffusion rates and also confer a significant element of overall chemical robustness (e.g. storability), when compared to the original ELISA test, based on conventional antibodies.

### Evaluation and Criteria for Acceptance:

The objective was to assess whether the final optimized P200 ModPro method shall provide identical results when benchmarked to the existing gold standard ELISA method on a reference set of 36 samples that previously also have been assayed by the reference lab at the Karolinska Institute. It was further desired to determine whether the final optimized P200 ModPro method shall minimally provide a comparable sensitivity and specificity when combined to the existing standard method., as the final optimized P200 ModPro method is a less complex, more robust, significantly faster (>2 h) and cheaper method.

### Results & Conclusions

Due to the limited number of variables to be explored it was decided to proceed with a full factorial design in three variables with two levels (2³ FD) plus three center points. The purpose of the latter experiments were partly to estimate the systematic variance between individual experimental runs, but also to evaluate the potential existence of nonlinearities in the response domain (i.e. interaction effects between variables). The variables identified for inclusion in the initial experimental design scheme and their corresponding settings and the first FD were thus defined as;

| ID | Name | Units | Low | Mid | High |
|---|---|---|---|---|---|
| V1 | ModPro concentration | [μg/ml] | 0.01 | 0.10 | 1.00 |
| V2 | Sample incubation time | [min] | 30 | 60 | 120 |
| V3 | BSA blocking time | [min] | 0 | 15 | 60 |

Full factorial 2³ with 3 center points

| Exp# | v1 | v2 | v3 |
|---|---|---|---|
| 1. | − | − | − |
| 2. | + | − | − |
| 3. | − | + | − |
| 4. | + | + | − |
| 5. | − | − | + |
| 6. | + | − | + |
| 7. | − | + | + |
| 8. | + | + | + |
| 9. | 0 | 0 | 0 |
| 10. | 0 | 0 | 0 |
| 11. | 0 | 0 | 0 |

The schema form above thus translates to the following 11 individual experiments:

| | [ModPro] | Binding | Blocking |
|---|---|---|---|
| | [μg/μl] | [min] | [min] |
| Exp 1. | 0.01 | 30 | 0 |
| Exp 2. | 1.00 | 30 | 0 |
| Exp 3. | 0.01 | 120 | 0 |
| Exp 4. | 1.00 | 120 | 0 |
| Exp 5. | 0.01 | 30 | 60 |
| Exp 6. | 1.00 | 30 | 60 |
| Exp 7. | 0.01 | 120 | 60 |
| Exp 8. | 1.00 | 120 | 60 |
| Exp 9. | 0.10 | 60 | 15 |
| Exp 10. | 0.10 | 60 | 15 |
| Exp 11. | 0.10 | 60 | 15 |

The results from the initial design clearly demonstrated that V1 was, by far, the single most influential factor to the sensitivity of the test, but also that the lowest blocking time (0 min) was too short in order to sufficiently inhibit unwanted background. Furthermore, the existence of any significant interaction effects between V1-V3, in the explored part of the response domain, could be excluded.

Although the response of the P200 ModPro method was significantly enhanced between the mid and high level setting, this increase was in practice less than optimal in two aspects. Firstly, the speed with which the reaction went to completion lead to a risk that tests become overexposed (i.e. reaching an absorbance of >2.0), i.e. a less robust method. Secondly, since the high level setting will consume more reactants and thus also lead to a more expensive test with lower margins.

The mid setting (0.10 m/ml) on the other hand allowed for a balance between workable reaction times and reasonable costs of reagents. The setting of ModPro concentrations was consequently fixed to this value in further experiments.

By employing the established protocol derived from the above multivariate approach, a validation experiment was performed on 36 clinical samples obtained from the Karolinska reference laboratory. The sample positioning scheme were as described in reference 3 and all samples and controls were run in duplicates. This scheme was repeated twice giving that each sample and control was run in total six times. One experiment was discarded when the ELISA washer flooded the plate, and was rerun at a later instance when the washer was corrected. The ModPro runs displayed a more even distribution of results than the previous ELISA runs (FIG. 4) and when compared with the Karolinska results also a more correct interpretation of the samples. There were some deviations noted between the Karolinska and the results of this assay. These major differences were addressed by asking Karolinska reference Laboratory to do reruns of the samples in question (six samples). After the Karolinska rerun all of the samples were either identical with the P200 ModPro runs or borderline interpretations with similar results.

With a second variant of the P200 ModPro, the BSA (bovine serum albumin) coupled ModPro, a limited number of samples were tested to evaluate if this variant was significantly better than the “naked” ModPro (data not shown). The results displayed virtually identical behavior of the ModPro-BSA as of the regular ModPro, hence no further testing was done on ModPro-BSA.

### Conclusions

The new P200 ModPro method works very well and meets all the objectives set forth above. The method is robust and gives reproducible and reliable results and has a significantly reduced operational time and a decreased number of steps. Furthermore, these results revealed that using six times less ModPro molecules compared to the ELISA antibodies still gave a stronger, more reliable and more robust signal. This finding demonstrates the power of this robust P200-sensing assay. Based on the findings set forth in this Example, the following protocol is recommended:

1. Add ModPro (0.1 μg/well) into the wells
2. Incubate 1 h at room temperature
3. Wash plate with PBS once.
4. Block with 4% BSA-PBS, 100 μL/well for 15 minutes
5. Wash with PBS once
6. Add samples and controls 100 μL/well
7. Incubate 1 h at room temperature on shaker
8. Wash 4 times with washing buffer

### 9. Add Conjugate 100 μL/well

10. Incubate Mon shaker
11. Wash 4 times with washing buffer
12. Add substrate 100 μL/well
13. Incubate 30 minutes
14. Read the plate in Elisa reader

### Example 3

The methods of the present invention, as set forth for example in Examples 1 and 2, will be used to assess the risk that a fetus will develop congenital heart block. Alternatively, the woman is not yet pregnant and the risk being assessed is whether she were pregnant whether the fetus would be at risk for developing congenital heart block. In either case an assay sample from a bodily fluid, such as serum, plasma, whole blood, or cerebrospinal fluid, is taken from the woman. For pregnant women, the source of this sample also includes amniotic fluid. The sample is then exposed to a vessel or scaffold, such as a well or dipstick, that has the RO52-antigen-containing ModPro sensor molecules. The sample is processed according to the methods set forth about in Examples 1 and 2 (a secondary antibody combined with a detection enzyme such as horseradish peroxidase can be used or the detecting/labeling molecule that is part of the ModPro sensor, such as a fluorophore, can be directly detected). Depending on the set-up, an increase or decrease in fluorescence intensity indicates an alternation and therefore binding of the ModPro sensor to an RO52-antibody. The presence of multiple RO52 antibodies can be assessed by having multiple RO52 antigen-ModPro sensor molecules present in the same assay.

The risk that a woman's fetus (or potential fetus) would develop congenital heart block can be determined by comparing the alternation in assay read out (such as fluorescence) with a control, known to lack RO52 antibodies. The risk can be further correlated by determining which of the many RO52 antibodies the woman has (by use, for example, of different colors for different RO52 ModPro antigens).

### Example 4

### A ModPro Multiplex Assay

Another embodiment of the present invention involves a multiplex version of the assays described herein. A multiplex assay is one in which a number of detection experiments are preformed in parallel. These parallel experiments are beneficial as the results from the different detection assays are directly comparable, since the assays are performed at the same time, using the same media, and often the sample source, therefore, offering the benefit of increased data utility. Multiplex assays also offer the benefit of higher throughput at a lower cost, since many experiments are being performed at the same time, thus reducing reagent and personnel costs.

One embodiment of the present invention is a multiplex assay involving two or more functionalized ModPro scaffolds. By way of example, a multiplex assay would involve the three ModPro sensor molecules p200 ModPro, p200-Q8/E28 ModPro, and p200-A25/S40 ModPro. Antibodies found to produce a greater response signal with the p200-Q8/E28 ModPro sensor, over the p200-A25/S40 ModPro sensor in the bodily fluid of a woman (a mother or potentially a mother), particularly a woman in need of screening, would indicate that a fetus of that woman would be at particular risk for developing congenital heart block (Salomonsson, S. et al., Ro/SSA autoantibodies directly bind cardiomyocytes, disturb calcium homeostasis, and mediate congenital heart block, J. Exp. Med., 2005, 201: 11-17).

### INDUSTRIAL APPLICATION

The precise detection, mapping and subsequent treatment of the disease states described above define a significant unmet medical need. A precise characterization of these disease states leading to the ability to subsequently administer adequate treatment will have a major economical and medical impact. A new diagnostic tool based on this invention, represents a fundamental improvement of standard clinical praxis.

From the foregoing it will be evident that, although specific embodiments of the invention have been described herein for purposes of illustration, various modifications may be made without deviating from the spirit and scope of the invention.

1. A method for assessing a risk that a fetus will develop congenital heart block comprising screening a bodily fluid of a woman in need of screening for the presence of an antibody specifically recognizing Ro52 p200 peptide, the method comprising: contacting a bodily fluid of the woman with an immobilized sensor molecule comprising a ModPro scaffold bearing a Ro52 p200 peptide and a fluorophore for a time period sufficient to allow any of said antibody that may be present in said fluid to bind to said molecule and thereby alter the fluorescence intensity of said fluorophore; and comparing the fluorescence intensity in said fluid after said time period to that of a control sample devoid of said antibody, wherein a difference in said fluorescence intensity correlates with said risk.

2. A method for detecting the presence of an antibody recognizing specifically Ro52 p200 peptide in a bodily fluid of a human comprising: contacting a bodily fluid of the human with an immobilized sensor molecule comprising a ModPro scaffold bearing a Ro52 p200 peptide and a fluorophore for a time period sufficient to allow any of said antibody that may be present in said fluid to bind to said molecule and thereby alter the fluorescence intensity of said fluorophore; and comparing the fluorescence intensity in said fluid after said time period to that of a control sample devoid of said antibody.

3. A method for assessing a risk that a fetus will develop congenital heart block comprising screening a bodily fluid of a woman in need of screening for the presence of an antibody specifically recognizing one or more Ro52 peptides, the method comprising: contacting a bodily fluid of the woman with a plurality of immobilized sensor molecules, each comprising a ModPro scaffold bearing an Ro52 peptide and a fluorophore for a time period sufficient to allow any of said antibody that may be present in said fluid to bind to said molecule and thereby alter the fluorescence intensity of said fluorophore; and comparing the fluorescence intensity of each sensor molecule after said time period to that of each of the other sensors.

4. A method according to claim 1, wherein the bodily fluid is serum.

5. A method according to claim 1, wherein the bodily fluid is plasma.

6. A method according to claim 1, wherein the bodily fluid is whole blood.

7. A method according to claim 1, wherein the bodily fluid is amniotic fluid.

8. A method according to claim 1, wherein the bodily fluid is cerebrospinal fluid.

9. A method according to claim 1, wherein the alteration in fluorescence intensity is an increase in fluorescence intensity.

10. A method according to claim 1, wherein the alteration in fluorescence intensity is a decrease in fluorescence intensity.

11. A method according to claim 1, wherein the Ro52 peptide has an amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, and SEQ ID NO: 18.

12. A method according to claim 2, wherein the bodily fluid is serum.

13. A method according to claim 3, wherein the bodily fluid is serum.

14. A method according to claim 2, wherein the bodily fluid is plasma.

15. A method according to claim 3, wherein the bodily fluid is plasma.

16. A method according to claim 2, wherein the bodily fluid is whole blood.

17. A method according to claim 3, wherein the bodily fluid is whole blood.

18. A method according to claim 2, wherein the bodily fluid is amniotic fluid.

19. A method according to claim 3, wherein the bodily fluid is amniotic fluid.

20. A method according to claim 2, wherein the bodily fluid is cerebrospinal fluid

21. A method according to claim 3, wherein the bodily fluid is cerebrospinal fluid

22. A method according to claim 2, wherein the alteration in fluorescence intensity is an increase in fluorescence intensity.

23. A method according to claim 3, wherein the alteration in fluorescence intensity is an increase in fluorescence intensity.

24. A method according to claim 2, wherein the alteration in fluorescence intensity is a decrease in fluorescence intensity.

25. A method according to claim 3, wherein the alteration in fluorescence intensity is a decrease in fluorescence intensity.

26. A method according to claim 2, wherein the Ro52 peptide has an amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, and SEQ ID NO: 18.

27. A method according to claim 3, wherein the Ro52 peptide has an amino acid sequence selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, and SEQ NO: 18.


SEQUENCE LISTING (18 sequences)

SEQ ID NO: 1 - protein, 475 aa, Homo sapiens [sequence omitted]

SEQ ID NO: 2 - protein, 42 aa, Artificial Sequence
  Description of Artificial Sequence Synthetic peptide
NAADXEAEIRHLREKXAARGPRDCAQXAEQLARRFERFARAG

SEQ ID NO: 3 - protein, 40 aa, Homo sapiens
LEKDEREQLRILGEKEAKLAQQSQALQELISELDRRCHSS

SEQ ID NO: 4 - protein, 40 aa, Artificial Sequence
  Description of Artificial Sequence Synthetic peptide
CEKDEREQLRILGEKEAKLAQQSQALQELISELDRRAHSS

SEQ ID NO: 5 - protein, 21 aa, Homo sapiens
QLRILGEKEAKLAQQSQALQE

SEQ ID NO: 6 - protein, 22 aa, Artificial Sequence
  Description of Artificial Sequence Synthetic peptide
CQLRILGEKEAKLAQQSQALQE

SEQ ID NO: 7 - protein, 16 aa, Homo sapiens
ALQELISELDRRCHSS

SEQ ID NO: 8 - protein, 17 aa, Artificial Sequence
  Description of Artificial Sequence Synthetic peptide
CALQELISELDRRAHSS

SEQ ID NO: 9 - protein, 36 aa, Homo sapiens
LQELEKDEREQLRILGEKEAKLAQQSQALQELISEL

SEQ ID NO: 10 - protein, 40 aa, Homo sapiens
LVKDLREQLRILGEKVAKLAQQSQALQELISELDRRCHSS

SEQ ID NO: 11 - protein, 40 aa, Homo sapiens
LEKDERQQLRILGNKEAKLAQQSQALQKLISELDRRCHSS

SEQ ID NO: 12 - protein, 40 aa, Homo sapiens
LEKDEREQLRILGEKEAKLAQQSQALQELISELARRCHSS

SEQ ID NO: 13 - protein, 40 aa, Homo sapiens
LEKDEREQLRILGEKEAKLAQQSQALQELISELDARCHSS

SEQ ID NO: 14 - protein, 40 aa, Homo sapiens
LEKDEREQLRILGEKEAKLAQQSQALQELISELDRACHSS

SEQ ID NO: 15 - protein, 40 aa, Homo sapiens
LEKDEREQLRILGEKEAKLAQQSQALQELISELDRRAHSS

SEQ ID NO: 16 - protein, 40 aa, Homo sapiens
LEKDEREQLRILGEKEAKLAQQSQALQELISELDRRCASS

SEQ ID NO: 17 - protein, 40 aa, Homo sapiens
LEKDEREQLRILGEKEAKLAQQSQALQELISELDRRCHAS

SEQ ID NO: 18 - protein, 40 aa, Homo sapiens
LEKDEREQLRILGEKEAKLAQQSQALQELISELDRRCHSA